(19) United States
(12) Patent Application Publication
(10) Pub. No.: US 2026/0195701 A1
(43) Pub. Date: Jul. 9, 2026

(54) SYSTEM AND METHOD FOR LOGISTICAL SUPPORT

(71) Applicants: Ivan Cardenas Bernal, Fremont, CA (US); Alberto Camarena Rodriguez, CDMX (MX); Carlos Alberto Velazquez Romero, CDMX (MX); Jose Ignacio Ambia Campos, CDMX (MX); Julio Cesar Tapia Guerrero, CDMX (MX); Federico Sanchez Gomez, CDMX (MX); Marcos Alan Gonzalez Schtulmann, State of Mexico (MX)

(72) Inventors: Ivan Cardenas Bernal, Fremont, CA (US); Alberto Camarena Rodriguez, CDMX (MX); Carlos Alberto Velazquez Romero, CDMX (MX); Jose Ignacio Ambia Campos, CDMX (MX); Julio Cesar Tapia Guerrero, CDMX (MX); Federico Sanchez Gomez, CDMX (MX); Marcos Alan Gonzalez Schtulmann, State of Mexico (MX)

(21) Appl. No.: 19/357,506

(22) Filed: Oct. 14, 2025

Related U.S. Application Data
(60) Provisional application No. 63/741,485, filed on Jan. 3, 2025.

(51) Int. Cl. G06Q 10/0834; G06Q 10/0832; G06Q 30/04; G06Q 50/26; G16H 80/00

(52) U.S. Cl. CPC G06Q 10/0834; G06Q 10/0832; G06Q 30/04; G06Q 50/265; G16H 80/00

(57) ABSTRACT

A system and method for logistical support is disclosed, addressing inefficiencies in data integration and analytics across industries such as logistics, trade, and finance. The disclosed system provides a unified platform that integrates diverse data sources, including geolocation systems, sensors, and transactional records, to optimize operations, enhance safety, and ensure compliance. Features include advanced modules for cargo matching, regulatory document generation, invoicing, funding solutions, medical services, fraud prevention, valuation, customs compliance, and fleet management. Machine learning algorithms analyze real-time data to generate actionable insights, automate processes, and improve decision-making. The system employs modular architecture, enabling scalability and adaptability, while offering tools such as predictive analytics, KPI dashboards, and geospatial visualizations. Primary uses include streamlining logistical operations, reducing manual intervention, and mitigating risks in dynamic environments. The disclosed system supports industries in achieving operational efficiency, regulatory adherence, and enhanced resource management.

### CROSS-REFERENCE TO RELATED APPLICATIONS

This application claims benefit to Provisional Application No. 63/741,485, filed Jan. 3, 2025, the contents of which are herein incorporated by reference.

### BACKGROUND OF THE INVENTION

### Field of Endeavor

The present disclosure pertains to data integration and analytics systems, specifically to systems and methods for collecting, processing, and utilizing industry-specific data to improve operations, promote safety, and support compliance in sectors such as logistics, trade, and finance.

### Background of Related Art

This disclosure relates generally to the field of data integration and analytics systems, with particular relevance to industries that rely on efficient logistical operations, trade facilitation, and financial processing. In today's digital age, businesses in sectors such as transportation, insurance, and finance are increasingly dependent on systems that combine information from diverse sources, including sensor networks, geolocation data, and transactional records. The drive toward digital transformation has led to an elevated need for platforms that can manage, correlate, and process large volumes of varied data in real time. This context underscores the importance of achieving seamless data synthesis to support accurate decision-making and enhanced operational performance.

A primary objective across these industries is to streamline operations by transforming disparate datasets into coherent, actionable insights. Enterprises are focused on solutions that minimize manual intervention, bolster safety protocols, and facilitate compliance with regulatory requirements. The overall aim is to establish systems that enable effective visualization, automated reporting, and real-time integration of significant data points. Such capabilities are necessary for optimizing asset allocation and resource management, thereby supporting ongoing efforts to improve operational efficiency and reduce process delays. Achieving these applicative goals remains a focal point in addressing the evolving challenges faced across multiple sectors.

While contemporary methods attempt to address the challenges through various means, many current approaches struggle with connectivity and cohesiveness. Systems that operate with isolated or siloed data tend to incur inefficiencies, often demanding extensive manual oversight. These disjointed approaches can compromise the accuracy and timeliness of critical decision-making processes, especially when rapid responses are needed in dynamic operational environments. The difficulties in unifying data flows from multiple inputs can result in suboptimal performance, thereby affecting operational safety and the effective management of resources.

More specifically, industries continue to grapple with the challenge of matching detailed operational requirements with the appropriate logistical resources. Manual efforts to correlate data—ranging from cargo specifications and transportation capacities to compliance documentation—are often labor-intensive and prone to error. The accumulation of diverse data types, including geospatial, sensor, regulatory, and financial information, complicates efforts to achieve a synchronized view of operations. This shortcoming not only hinders timely decision-making, but also raises concerns about operational efficiency, safety, and risk management in high-stakes environments, highlighting a clear need for more integrated and adaptive solutions.

### SUMMARY OF THE INVENTION

In one embodiment, a system for logistical support includes a processing device, a communication device for receiving logistical support data from one or more data sources, and a memory device storing an interface module, a storage module, and a Logistical Support module. The interface module provides a graphical user interface or application programming interface for ingesting data items and presenting outputs. The Logistical Support module comprises multiple sub-modules—cargo matching, regulatory document generation, invoicing, funding determination, medical services enrollment, compliance monitoring, valuation, customs formatting, cargo evaluation, route generation, classification, reporting, and fleet management. The processing device is configured to receive and store the logistical data, generate pairing recommendations between shippers and carriers via one or more machine learning models, invoke the appropriate sub-modules in real time based on user selection, and output the results through the interface.

The cargo matching sub-module generates recommendations using a mixed integer linear programming model or predictive scores from a supervised machine learning model; the regulatory sub-module automatically produces shipment documents such as Carta Porte or Bill of Lading and validates them against schemas retrieved from external repositories; the funding sub-module combines a dynamic scoring model with a rules-based trust engine and interfaces with external financial platforms to disburse funds; the medical sub-module integrates with telemedicine services and wearable devices for real-time health monitoring; the compliance sub-module employs unsupervised learning on historical transactions and reinforcement learning with human feedback to detect anomalies; the valuation sub-module applies statistical anomaly detection against internal and external benchmarks; and the routing sub-module computes a dynamic crime risk index for each route segment based on historical crime data, time-of-day sensitivity, and environmental conditions.

In a further embodiment, a method for providing logistical support is performed by a computing system comprising a processor, a communication interface, and a non-transitory storage medium. The method comprises receiving logistical support data, generating carrier pairing recommendations via machine learning, and, upon user selection of a recommendation, performing in real time one or more functions selected from: generating regulatory shipment documents; producing invoices; determining funding solutions; enrolling carriers in insurance plans and delivering medical services; detecting and preventing compliance issues; determining monetary valuations of cargo; formatting customs information for cross-border compliance; evaluating cargo for hazardous materials and handling requirements; generating and evaluating shipment routes based on geospatial data and risk assessments; classifying goods using standardized codes; synthesizing visual reports; and monitoring and optimizing carrier fleet performance. The data may include cargo details, vehicle capacity, geolocation and sensor readings, and the machine learning models may comprise mixed integer linear programming or supervised learning approaches.

### BRIEF DESCRIPTION OF THE DRAWINGS

FIG. 1 is a schematic diagram of a System for Logistical Support, according to aspects of the present invention;

FIG. 2 is a flow diagram of a Method for Logistical Support, according to aspects of the present invention;

FIG. 3 is a flow diagram of an exemplary Method of Order Placement, according to aspects of the present invention; and

FIG. 4 is a flow diagram of a Method of an Artificial Intelligence Virtual Assistant, according to aspects of the present invention.

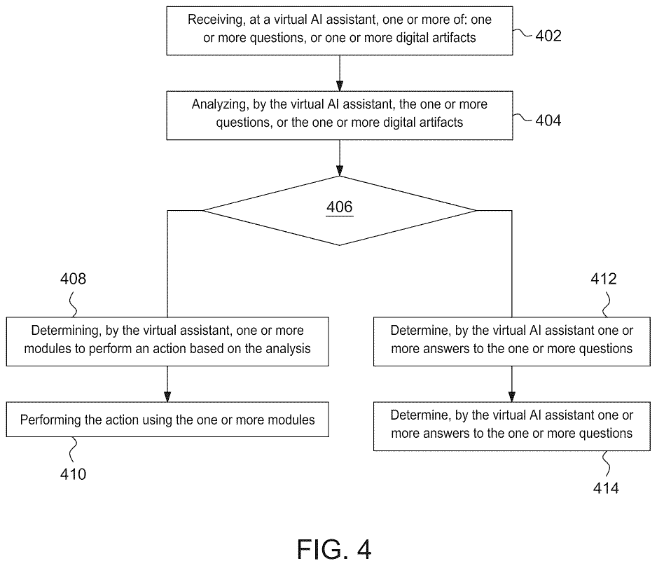

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Industries such as logistics, trade, insurance, and finance encounter significant challenges in managing and utilizing diverse datasets to optimize operations, ensure compliance, and enhance decision-making. Existing systems often function in isolation, relying on disconnected data sources and manual processes that result in inefficiencies, errors, and delays. For instance, matching cargo with carriers, generating regulatory documents like Carta Porte, and automating invoicing frequently demand extensive manual intervention, which is labor-intensive and prone to inaccuracies. Additionally, current solutions face difficulties in providing real-time insights, integrating advanced analytics, or addressing issues such as fraud prevention, undervaluation detection, and safety monitoring. These constraints impede operational efficiency, compromise safety, and create obstacles to scalability and adaptability in dynamic environments.

The present system and method address these shortcomings by introducing a unified approach for collecting, processing, and utilizing industry-specific data through advanced integration of diverse data sources. The described approach employs specialized algorithms, artificial intelligence (AI), and machine learning (ML) models to aggregate and analyze data from geolocation systems, sensors, relational and non-relational databases, and user inputs. By creating a cohesive data ecosystem, the system facilitates seamless automation of processes such as cargo matching, regulatory compliance, invoicing, and financial resource allocation. For example, the system efficiently matches cargo with carriers based on real-time availability, capacity, and location, while automating the generation of critical documents like Carta Porte and customs tapes to ensure adherence to regulatory standards.

Additionally, the system integrates advanced visualization tools, including geospatial maps, predictive analytics, and KPI dashboards, to provide actionable insights that facilitate data-driven decision-making. Safety and risk management are improved through features such as telemedicine services for drivers, fraud detection algorithms, and real-time monitoring of hazardous cargo. The platform also accommodates personalized applications, such as tailored medical insurance plans for logistics workers, and addresses broader challenges like tax fraud prevention and undervaluation detection by analyzing and correlating diverse datasets.

By integrating these components into a scalable, modular architecture, the described system overcomes the limitations of conventional approaches, offering a robust, adaptive solution that optimizes operations, enhances safety, ensures compliance, and supports decision-making across multiple industries. This comprehensive framework not only reduces inefficiencies but also provides a foundation for future advancements, such as blockchain integration for secure data sharing or enhanced AI models for real-time risk mitigation.

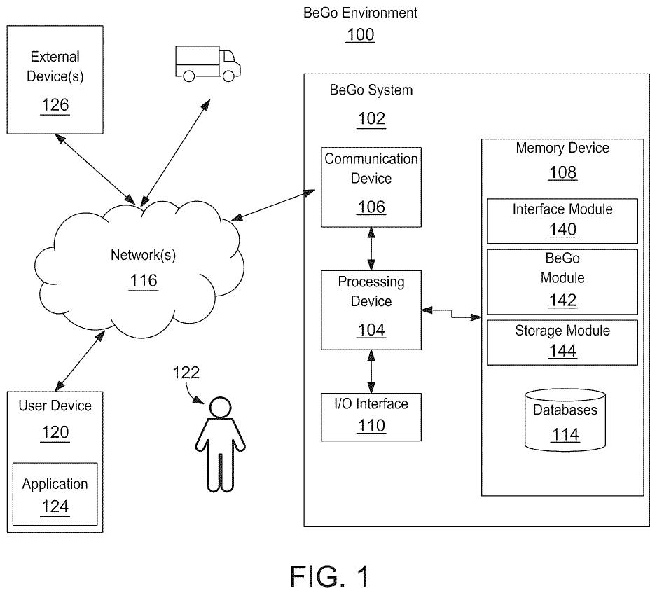

FIG. 1 illustrates a BeGo Environment 100, according to aspects of the present disclosure. While FIG. 1 illustrates various components of the BeGo Environment 100, additional components can be added, and existing components can be removed.

As illustrated in FIG. 1, the BeGo System 102 includes one or more processing devices, herein processing device 104, coupled to a communication device 106. The processing device 104 is also coupled to a memory device 108, and an input/output (“I/O”) interface 110. In embodiments, the communication device 106 enables the BeGo System 102 to communicate with other devices and systems via one or more networks 116. The BeGo System 102 can communicate with a user device 120 via the network 116. A user 122 can utilize the user device 120 to communicate with the BeGo System 102. The user device 120 can include one or more electronic devices such as a laptop computer, a desktop computer, a tablet computer, a smartphone, a thin client, a smart appliance, and the like. While FIG. 1 illustrates one user device 120, the network trading environment 100 can include multiple user devices operated by the user 122 or operated by other users.

According to the aspects of the present disclosure, the BeGo System 102 enables the user 122, operating a copy of an application 124 executing on the user device 120, to communicate with the BeGo System 102 and leverage the service provided by the BeGo System 102. The BeGo System 102 is configured to perform one or more logistical support functions associated with shipping and receiving of goods.

To perform the process described herein, the BeGo System 102 can store and execute an Interface Module 140, a BeGo Module 142, and an Storage Module 144 to perform the processes and methods described herein. The Interface Module 140, the BeGo Module 142, and the Storage Module 144 can be stored in the memory device 108. The Interface Module 140, the BeGo Module 142, and the Storage Module 144 can include the necessary logic, instructions, and/or programming to perform the processes and methods described in further detail below. The Interface Module 140, the BeGo Module 142, and the Storage Module 144 can be written in any programming language.

In embodiments, the application 124 can be a specifically designed application that operates with the BeGo System 102 to perform the processes and methods described herein. In embodiments, the application 124 can be a third-party application, such as a web browser, that communicates with the BeGo System 102 to perform the processes and methods described herein. The memory device 108 can also include one or more databases 114 that store information and data associated with the process and methods described below in further detail.

According to aspects of the present disclosure, the BeGo System 102, for example, via the Interface Module 140, provides unique interfaces that allow the user 122 view, updated, modify, perform or otherwise interact with one or more components of BeGo System 102. The Interface Module 140 operates to generate and provide graphical user interfaces (GUIs) to the application 122, for example, menus, widgets, text, images, fields, etc., as described below in further detail. The GUIs generated by the Interface Module 140 can be interactive. The BeGo System 102, for example, via the Interface Module 140, also provide one or more application programming interface (APIs) that provide connection points for one or more application, e.g., the application 124.

In embodiments, the Interface Module 140 can implement voice control aspects into the interfaces provided. For example, the user can navigate the interfaces of the BeGo System 102 using the audio input device of the user device 120. The interface module 140 can implement one or more chat-bots to deliver conversational input and output to a user.

According to aspects of the present disclosure, the BeGo System 102, for example, via the BeGo Module 142, provides functionalities associated with Logistic Support, using one or more sub-modules, processes, threads, applications, etc. In embodiments, the one or more sub-modules of BeGo Module 142 can include, but are not limited to, a cargo sub-module configured to match one or more cargo shippers with one or more cargo carriers, a regulatory sub-module configured to automatically generate one or more documents needed for cargo shipping, an invoice sub-module configured to automate financial transactions between the one or more cargo shippers and the one or more cargo carriers, a funding sub-module configured to provide funding solutions for the one or more cargo carriers, a medical sub-module configured to provide one or more medical services and/or insurance services to the one or more cargo carriers, a compliance sub-module configured to analyze and prevent one or more compliance issues, a valuation sub-module configured to analyze and value goods in cargo, a customs sub-module configured to generate standardized cargo information for compliance when shipping across one or more jurisdictional borders, a cargo evaluation sub-module configured to evaluate cargo details, a routing sub-module configured to generate and evaluate routes for the one or more cargo carriers, a classification sub-module configured to classify cargo, a report sub-module configured to provide one or more reports using information from one or more of the sub-modules, and/or a fleet management sub-module configured to manage one or more carrier fleets.

In embodiments, the cargo sub-module is configured to match one or more shippers to one or more carriers. The cargo sub-module collect(s) one or more data items as inputs, such as, but not limited to geolocations of a shipper or a carrier, vehicle capacity, available cargo space, cargo type, and/or cargo specific details, such as weight, dimensions, handling requirements, etc. In embodiments, the one or more data items are collected in real-time, or alternatively, can be collected and stored in databases 114 and provided to cargo sub-module using Storage Module 144. In addition to the one or more data items, inputs such as historical performance metrics, such as carrier reliability and/or efficiency can be provided as inputs, and outputs of the one or more sub-modules of Cargo Module 142 can be provided as inputs.

The cargo sub-module analyzes the one or more data items, such as carrier availability, vehicle capacity, shipment characteristics, and/or historical performance indicators, to determine one or more optimal pairings between the one or more shippers and the one or more carriers, in real-time. In embodiments, the one or more data items are input into one or more optimization algorithms, and/or one or more machine learning algorithms, to determine the one or more optimal pairings. In embodiments, the one or more optimization algorithms process the one or more data items to minimize and/or maximize one or more constraints, such as empty cargo mileage, delays, costs, and/or overall efficiency. In an exemplary embodiment, the one or more optimization algorithms can include linear programming, and/or heuristic algorithms. For example, a mixed-integer linear programming model incorporating constraints such as maximum weight, delivery windows, and geographic distance can be utilized for optimization. The mixed integer linear programming model is further enhanced with predictive scores generated by a supervised machine learning model trained on prior shipment records to evaluate carrier reliability and timeliness. Once candidate pairings are produced, the system verifies mandatory shipment requirements—such as refrigeration or proximity thresholds—using a rule-based validation engine. The result is a shortlist of pairing recommendations optimized for efficiency, compliance, and service quality, ready to be reviewed or automatically approved by dispatch logic, and subsequently propagated across downstream systems for execution.

Additionally, the one or more data items are input into one or more machine learning algorithms configured to analyze historical patterns and predict a success/failure of one or more pairings. Finally, the one or more data items can be analyzed by one or more rule-based filters configured to guarantee one or more requirements, specified in the one or more rule-based filters is met. In an exemplary embodiment, rules-based filters can utilize geospatial information to ensure carriers and shippers in close proximity are matched, and/or one or more cargo requirements is met, such as refrigeration required.

The cargo sub-module outputs one or more pairing recommendations between the one or more shippers and the one or more carriers in real-time, for consideration and selection by the one or more shippers and/or the one or more carriers. In embodiments, the one or more pairing recommendations can include one or more details, such as, but not limited to, optimal carrier options for a given shipment, estimated transportation costs and delivery times, and/or a compliance status of any given pairing. In embodiments, the one or more shippers or the one or more carriers, can review these recommendations and proceed with their preferred choices or negotiate terms as needed. In embodiments, once a match is selected and cargo is delivered, the cargo sub-module can collect one or more feedback items from the one or more shippers and/or the one or more carriers, such as delivery time, cargo conditions upon delivery, and/or overall satisfaction. The one or more feedback items is fed into the one or more machine learning algorithms to continuously refine and improve an accuracy and performance of the algorithms, with respect to pairing determination.

Advantageously, the cargo sub-module serves as a dynamic connector between shippers and carriers. By leveraging real-time data and AI-powered algorithms, this module ensures efficient logistics operations by identifying optimal pairings based on several key factors.

In embodiments, the regulatory sub-module configured to automatically generate one or more documents needed for cargo shipping. The regulatory sub-module collect(s) one or more data items as inputs, such as, but not limited to cargo details, such as weight, dimensions, specific handling requirements, etc. Additionally, the one or more data items can include geolocation data to accurately define a route, including, but not limited to, origin, destination, and/or any intermediate waypoints. Finally, the one or more data items can include vehicle specifications, such as license numbers, capacity, and compliance certifications. In embodiments, the one or more data items are collected in real-time, or alternatively, can be collected and stored in databases 114 and provided to regulatory sub-module using Storage Module 144.

The regulatory sub-module utilizes the one or more data items to generate one or more regulatory documents needed for shipping, such as a Carta Porte, or Bill of Lading. In embodiments, the regulatory sub-module automatically generates the one or more regulatory documents using all, or a portion of the one or more data items input into the regulatory sub-module. In embodiments, regulatory sub-module automatically generates one or more Carta Porte, or Bill of Lading, by extracting from the one or more data items, cargo details, route information, vehicle specifications, etc., and entering the information into one or more digital forms.

The regulatory sub-module validates the one or more regulatory documents against legal and regulatory standards, using one or more Advanced algorithms, In embodiments, the one or more advanced algorithms can identify and/or remediate any discrepancies in the one or more regulatory documents.

In embodiments, the regulatory sub-module fetches one or more validation schemas for the one or more regulatory documents. The one or more validation schemas are fetched from one or more repositories, such as database 114, and/or external repositories, such as government and/or regulatory repositories. The one or more validation schemas are utilized by the regulatory sub-module to validate the one or more documents, i.e. determining compliance with the one or more validation schemas. Any validation error determined by the one or more validation schemas are returned for manual review, and/or correction. In embodiments, the one or more regulatory documents are dynamic documents that must be altered during shipping, the regulatory sub-module interfaces with one or more additional modules to receive real-time information items, and update the one or more regulatory documents accordingly. For example, to maintain up-to-date accuracy, the regulatory sub-module consumes events, webhooks, WebSocket notifications, REST callbacks, or periodic polling from routing, cargo, and other related components; whenever new data arrives, the module automatically revisits and updates the affected one or more documents so that they remain fully compliant throughout the shipping process. Advantageously, the regulatory sub-module ensures all necessary regulatory documentation is prepared accurately and efficiently, supporting seamless cargo transport operations while maintaining full compliance with governmental requirements.

In embodiments, the invoice sub-module configured to automate financial transactions between the one or more cargo shippers and the one or more cargo carriers. The invoice sub-module is designed to automate financial transactions between the one or more shippers and the one or more carriers, ensuring seamless and efficient payment workflows. This sub-module integrates with other components of the system, such as the cargo sub-module and the regulatory sub-module, to create a cohesive financial process.

The invoicing sub-module collects data from various sources, including: service details such as shipment ID, carrier details, delivery timelines, and agreed-upon rates, from the cargo sub-module; Compliance-related data, such as Carta Porte or other regulatory documents, to verify that all legal and logistical requirements have been met from the regulator sub-module; and/or one or more user inputs such as additional financial details, such as payment terms, applicable taxes, and fees.

Once confirmation of a match between the one or more shippers and the one or more carries in the cargo sub-module, the invoice sub-module automatically generates one or more invoices using predefined templates that include all relevant financial and service details. The one or more invoices are tailored to the specifics of the transaction (shipment), incorporating elements such as a shipment ID, carrier details, cargo specifications, and applicable taxes or fees. The invoice sub-module employs algorithms to calculate totals based on the terms of the agreement, including any additional costs or adjustments.

In addition to invoice generation, the invoice sub-module streamlines payment workflows by integrating with one or more external financial systems, such as accounting software or payment gateways, to automate the transfer of funds. For example, once an invoice is approved, the invoice sub-module initiates a payment requests or track payment statuses, ensuring that transactions are processed promptly and efficiently.

The invoicing sub-module produces detailed financial documents that include all relevant information, such as shipment details, costs, and compliance data, as invoices; Initiates payment workflows through integration with external financial systems or payment gateways; and/or maintains a comprehensive record of all transactions, linking service details to financial records for transparency and traceability.

In an exemplary embodiment, the invoicing sub-module generates financial documents by aggregating shipment metadata, carrier service details, and validated regulatory records into standardized invoice templates compatible with third-party accounting platforms. Once an invoice is finalized, the module initiates the payment process by invoking a financial API exposed by the selected external payment or ERP system. Coordination between systems is managed via SDK-level integrations using webhooks and event-driven triggers, ensuring timely processing and error tracking. All invoice transactions, including audit trails and payment states, are stored in a tamper-proof ledger structure to guarantee both immutability and traceability, enabling comprehensive compliance and financial reporting across the operation.

In embodiments, the funding sub-module configured to provide funding solutions for the one or more cargo carriers. The funding sub-module receives one or more inputs from the cargo sub-module, such as availability, job details, and pricing agreements information, and one or more inputs from the invoice sub-module, such as invoicing and/or billing information, ensuring a linkage between potential funding offerings and eventual payments received.

The funding sub-module analyzes the one or more inputs from the cargo sub-module and/or the invoice sub-module and determines an appropriate funding amount, based on the inputs. In embodiments, one or more risk assessment algorithms can take one or more of the inputs, such as carrier past performance, payment histories of the shipper, and/or expected revenue from the prospective job, and provide a suggested or recommended funding amount based on the results of the analysis. In embodiments, the funding sub-module integrates with external financial systems and payment platforms, providing the suggested funding amount to the external financial systems and payment platforms, which in turn can make one or more funding options available to the one or more carriers. In embodiments, the external financial systems and payment platforms offer the entire suggested amount, or a portion thereof, which can be accepted, or rejected, by the one or more carriers seeking funding.

In an exemplary embodiment, the funding sub-module evaluates funding eligibility by combining a dynamic scoring model with a rules-based trust engine that reflects the user's historical behavior and activity within the platform. This includes factors such as successful delivery rates, invoice payment timelines, and platform tenure. Based on this evaluation, the system computes a funding amount aligned with the user's operational reliability and prior engagement. Rather than issuing the offer automatically, the calculated suggestion is forwarded to a user, such as an internal sales user, to review the context and prepare tailored proposals. Once a proposal is approved and the user initiates a new service that qualifies for financing, the system triggers the funding offer and notifies the user directly, integrating the event into the transaction flow for seamless tracking.

The funding sub-module includes integrated payments, such that one or more carriers receive funding from the external financial systems and payment platforms through a unified interface. Additionally, the funding sub-module maintains one or more record(s) of all transactions, creating a transparent and traceable system for managing financial instruments. These record(s) link funding disbursements to specific jobs, ensuring that the system remains accountable and providing a clear audit trail for all parties involved.

By offering upfront funding, this module helps carriers overcome cash flow challenges, enabling them to accept more jobs and maintain reliable service. It also enhances the overall efficiency of the system by reducing delays caused by financial constraints, creating a more resilient and responsive logistics network. Through its integration with other components, this module plays a vital role in ensuring that operational and financial workflows remain seamlessly connected.

In embodiments, the medical sub-module configured to provide one or more medical services and/or insurance services to the one or more cargo carriers. The medical sub-module receives one or more user profile information related to one or more cargo carriers, and/or their dependents, and/or one or more insurance details, such as policy terms, covered services, beneficiaries, etc., for use in enrollment in one or more health insurance plan(s). In embodiments, enrollment is automated by the medical sub-module allowing the enrollee to select one or more health insurance plans based on the one or more insurance details provided. The medical sub-module tracks the one or more insurance details, providing the enrollees with a clear understanding of benefits provided.

The medical sub-module, in addition to enrollment, is configured to provide access to one or more medical services and/or information, such as telemedicine, distribute health education materials, reminders for routine check-ups, and wellness tips tailored to the physical demands of driving. Enrollees, such as drivers, can schedule virtual consultations with licensed medical professionals for immediate health concerns. This functionality is particularly advantageous for drivers operating on long highways or in rural areas where access to hospitals or doctors may be limited. By leveraging telecommunication technologies, the medical module ensures that drivers can address minor health issues before they escalate, minimizing disruptions and promoting overall well-being. Additionally, the medical sub-module integrates with wearable devices or health apps to monitor drivers' vital signs, offering real-time alerts in case of emergencies such as elevated blood pressure or fatigue.

In an exemplary embodiment, the medical sub-module facilitates access to both in-person and virtual healthcare services through established partnerships with external health providers. Users enrolled through the platform can request medical assistance, and when eligible, are redirected to a partnered telemedicine service for virtual consultations. Appointment scheduling is initiated from within the platform, with user identity and context pre-filled to streamline the process. All personal health-related interactions are handled securely, with the platform serving as a gateway—not a repository—for medical data. This integration allows logistics workers, especially those in remote or mobile conditions, to receive timely medical attention with minimal disruption to their routes or operations.

To support its functionality, the medical module can interface with other components of the system 102. For instance, it can use geolocation data to recommend nearby clinics, pharmacies, or emergency services, ensuring that drivers have access to in-person care when necessary. It can also link with funding or invoice sub-modules to manage insurance claims and reimbursements, streamlining the financial aspects of healthcare access.

In embodiments, the compliance sub-module configured to analyze and prevent one or more compliance issues, such as issues of fraud. The compliance sub-module interfaces with the regulatory sub-module and the invoice sub-module to identify and prevent fraudulent activities in the shipping/carrier logistics chain. In embodiments, one or more fraud prevention algorithms in the compliance sub-module are configured to detect and prevent fraud by analyzing invoicing and regulatory data for anomalies, thereby ensuring transparency and accuracy.

In embodiments, the one or more fraud prevention algorithms collect one or more information items related to one or more shipping orders, such as data from the regulatory module like a Carta Porte document, and cross-reference the one or more information items with financial records from the invoice sub-module, including invoices and payment details. By correlating these datasets, the module creates a comprehensive picture of the transactions and compliance status within the logistics chain.

In an exemplary embodiment, the compliance sub-module is designed to detect potential fraud by analyzing patterns across invoicing and regulatory documentation using a layered detection strategy. At its core, the compliance sub-module employs an unsupervised learning model trained on transactional data to identify statistical outliers in billing behavior, document correlation, and cargo classification. These results are then refined using reinforcement learning with human feedback (RLHF), enabling the model to continuously improve by learning from decisions made by the operations and compliance teams. This iterative feedback loop sharpens the model's sensitivity to new and evolving fraud behaviors without sacrificing accuracy. When discrepancies—such as mismatched cargo descriptions, duplicate invoices, or irregular billing frequencies—are detected, the sub-module flags the cases and sends real-time alerts to the operations dashboard and the appropriate internal teams. This process ensures that all potential issues are visible and actionable while preserving human oversight for final resolution.

In embodiments, the one or more fraud detection algorithms rely on machine learning models trained on historical data to identify patterns that deviate from normal operations. For example, the one or more fraud detection algorithms, using machine learning modules, flag discrepancies such as mismatched cargo details between invoices and regulatory documents, inflated or duplicate billing, or missing regulatory information. The algorithms also analyze transaction sequences to detect irregularities, such as payments to unverified accounts or unexpected changes in tax calculations.

Once potential fraud is identified, the compliance sub-module triggers alerts to notify relevant stakeholders, such as shippers, carriers, or auditors. These alerts are accompanied by detailed reports outlining the anomalies detected, the associated risks, and suggested actions to address the issue. In more advanced configurations, the compliance sub-module takes proactive steps, such as temporarily halting suspicious transactions or flagging them for manual review.

To enhance its effectiveness, the machine learning models of the compliance sub-module continuously learns from new data and updates to improve its detection capabilities over time. Furthermore, the compliance sub-module integrates seamlessly with external auditing tools and tax compliance systems to ensure alignment with legal standards. By automating the detection and prevention of fraud, the compliance sub-module not only reduces financial losses but also fosters trust and accountability within the logistics chain. It ensures that all transactions and operations are transparent, accurate, and compliant with fiscal regulations, strengthening the overall integrity of the system.

In embodiments, the valuation sub-module configured to analyze and value goods in cargo. The valuation sub-module receives one or more inputs from the cargo sub-module and the regulatory sub-module and analyzes the inputs to determine one or more classifications and/or monetary valuations of cargo.

The valuation sub-module can include one or more valuation algorithms, such as statistical anomaly detection algorithms and/or machine learning models, which are trained to recognize patterns indicative of undervaluation, such as unusually low declared values for specific types of goods or discrepancies between the cargo's declared value and its logistical attributes (e.g., size or weight). The module may also incorporate geospatial and market data to refine its assessments, accounting for regional price variations or current market trends.

In an exemplary embodiment, the valuation sub-module estimates the expected value of cargo by comparing declared figures against internally generated reference pricing benchmarks. These benchmarks are maintained through a hybrid strategy: aggregating historical platform declarations and periodically synchronizing with external customs valuation databases and pricing APIs. The valuation sub-module applies statistical anomaly detection to identify declared values that fall significantly outside expected ranges for a given cargo type, weight, and dimension. Additionally, rule-based logic checks for consistency across classification codes, logistical parameters, and declared amounts to ensure structural coherence in reported values. When a potential undervaluation is identified, the sub-module routes the case to a valuation dashboard with visual context and supporting data, allowing designated personnel to assess the issue and take corrective action if needed. This process helps prevent tax-related violations while reinforcing the integrity of shipping documentation.

The one or more valuation algorithms utilize the one or more inputs, such as, cargo details, including weight, dimensions, declared value, and type of goods. The one or more inputs are cross-referenced with regulatory benchmarks, such as standardized valuation guidelines, tariff classifications, and historical pricing data for similar shipments. By comparing the declared value of goods with these benchmarks, the valuation sub-module identifies potential instances of undervaluation or misclassification.

When undervaluation is detected, the valuation sub-module generates alerts detailing the discrepancies, the potential financial impact, and the steps required to correct the valuation. These alerts are shared with relevant stakeholders, such as customs officials, shippers, or carriers, enabling them to address the issue promptly. In more sophisticated configurations, the valuation sub-module can take proactive measures, such as flagging shipments for further inspection or temporarily halting documentation processes until the valuation is rectified.

To ensure accuracy and compliance, the valuation sub-module continually updates its valuation criteria and algorithms based on regulatory changes and feedback from previous analyses. Additionally, it integrates with external tax and customs systems, enabling seamless validation and reporting of valuation data. Advantageously, by preventing undervaluation, the valuation sub-module safeguards tax revenue and promotes fair trade practices within the logistics chain and ensures that all cargo is properly classified and valued, reducing the risk of financial penalties, legal disputes, and operational delays.

In embodiments, the customs sub-module configured to generate standardized cargo information for compliance when shipping across one or more jurisdictional borders.

The customs sub-module collects relevant shipment data, including product descriptions, values, weights, and origin, as well as any other specific customs requirements, from one or more sub-modules such as the cargo sub-module and/or the regulatory sub-module. In embodiments, one or more data transformation functions transforms the collected shipment data to one or more standardized formats required by customs authorities. In embodiments, the one or more data transformation functions are configured to transform the collected shipment data based on a shipment destination jurisdiction, and/or are configured to provide one or more universal data transformation applicable to any jurisdiction. In embodiments, the one or more data transformation functions can transform the collected shipment data and transform the data such that shipment descriptions align with global customs codes, such as Harmonized System (HS) codes, and meet regulatory standards across different regions.

In an exemplary embodiment, the customs sub-module prepares cargo information for cross-border compliance by aggregating shipment data from upstream modules and converting it into jurisdiction-specific digital formats. This process includes mapping local product descriptions and logistics metadata to global customs frameworks such as Harmonized System (HS) codes, tariff identifiers, and declared values. The transformation logic adapts dynamically based on the destination jurisdiction by applying rule sets defined per country or trade block, ensuring that documentation conforms to the respective electronic declaration schema—typically using structured JSON or XML formats. The sub-module also verifies completeness and field accuracy prior to submission, and when required, applies a generalized transformation engine to align shipments with multilateral customs agreements. Once the transformation is complete, the resulting structure is made available for regulatory systems or export through the platform's integration interface.

Additionally, the customs sub-module can utilize machine learning models trained on historical customs data to identify potential discrepancies or risks. For example, if a shipment's description does not match its declared value or weight, the module may flag this as a potential compliance issue. The customs sub-module is also configured to cross-reference the shipment data, such as cargo details with applicable trade agreements, tariff rules, and customs regulations to ensure that the correct duties, taxes, and other requirements are applied.

The customs sub-module is configured to output one or more standardized customs documents or data structures that are formatted to meet one or more jurisdiction's customs requirements. This includes a complete customs declaration, along with any necessary compliance reports. If any discrepancies are identified, the module generates an alert indicating the issue, prompting corrective actions to be taken. Advantageously, by automating and standardizing customs documentation, this module helps to streamline the shipping process, reduce human error, and minimize the risk of customs-related delays or penalties. It ensures that all necessary information is presented accurately and in accordance with the relevant regulations, promoting smooth cross-border trade.

In embodiments, the cargo evaluation sub-module configured to evaluate cargo details. The cargo evaluation sub-module evaluates cargo descriptions to identify hazardous materials or special requirements, facilitating safety and compliance.

The cargo evaluation sub-module can receive one or more inputs from such as cargo details, including descriptions, materials, weight, and shipping conditions. In embodiments, a Natural language processing (NLP) engine is configured to analyze the one or more inputs the for key terms or phrases that indicate the type of goods, such as “flammable,” “toxic,” or “perishable.” In embodiments, the key terms and/or phrases determined by the NLP engine are used by the cargo evaluation sub-module to categorize the goods, in the cargo shipment, into predefined categories, such as hazardous materials or temperature-sensitive goods, based on regulatory standards, which can be used to further process cargo.

In embodiments, if cargo is classified as hazardous materials, the cargo evaluation sub-module applies decision trees or rules-based systems to assess the risk level associated with each cargo item. The cargo evaluation sub-module uses data from regulatory bodies, such as the United Nations'classification system for hazardous goods, to classify materials based on their safety requirements. Additionally, one or more machine learning models can be applied to predict the risk level of cargo, accounting for factors like its potential impact on safety, the environment, and personnel. In embodiments, cargo is classified as requiring special handling requirements, such as refrigeration or secure storage, based on the classification of the cargo.

The cargo evaluation sub-module is configured to output one or more detailed cargo classification report(s), which provides a clear indication of the cargo's type, potential hazards, and any special handling instructions. The one or more report(s) helps shippers, carriers, and customs authorities ensure that the cargo is appropriately handled and meets all safety and regulatory requirements.

In embodiments, the routing sub-module configured to generate and evaluate routes for the one or more cargo carriers. The routing sub-module is configured to gather geospatial data, including the origin, destination, and waypoints along one or more routes for a given shipment. The route sub-module analyzes the geospatial data in conjunction with one or more additional data items, such as historical crime statistics, including theft, hijacking, or other criminal activities, to assess the risk associated with specific routes or regions. In embodiments, and additionally, one or more Machine learning algorithms are applied to predict the likelihood of criminal activity along a given route, considering factors like time of day, local crime trends, and environmental conditions.

In an exemplary embodiment, the routing sub-module generates intelligent shipment paths by combining geospatial data with public crime datasets and historical incident records gathered from logistics partners. Each proposed route is evaluated using a scoring model that quantifies exposure to risk based on location-specific crime density, time-of-day sensitivity, and cargo classification. The scoring model computes a dynamic crime risk index that weighs the likelihood of theft, hijacking, or other security events along each segment of the path. The final route output includes both the optimal logistical path and a breakdown of risk levels across regions, enabling dispatchers to choose safer alternatives or apply additional countermeasures. Risk indices and suggested actions are surfaced directly in a planning interface to support proactive operational decisions.

The routing sub-module is configured to output at least one crime risk index for each of the one or more routes, highlighting areas with high-risk potential. The routing sub-module, using the at least one crime risk index, can suggest one or more optimal routes, and/or provide one or more real-time alerts if a shipment is at risk of encountering criminal activity. This enables logistics companies to take proactive measures, such as adjusting routes or providing additional security, to ensure the safety of goods and personnel. By integrating these analyses, the module enhances the overall security of the transportation network, reducing the risk of theft, hijacking, or other criminal disruptions. It ensures that shipments are routed safely, providing peace of mind for stakeholders and improving the efficiency of logistics operations.

In embodiments, the classification sub-module configured to classify cargo. The classification sub-module receives one or more classification inputs from one or more sub-modules, such as the cargo sub-module and/or the regulatory sub-module. In embodiments, the one or more classification inputs can include one or more cargo details, such as cargo details like product descriptions, product weights, and product quantities.

The classification sub-module utilizes one or more classification algorithms, such as rule based classification algorithms, and/or machine learning classification algorithms, which rely on known classification schemes, such as Homeland Security (HS) codes and Tarriff of the General Import and Export Tax Law (TIGIE) classifications, to match one or more classification inputs to one or more known standards to properly classify cargo.

In an exemplary embodiment, the classification sub-module standardizes cargo descriptions by automatically mapping shipment data—such as product names, categories, quantities, and dimensions—to international classification standards like HS codes and the TIGIE taxonomy. The classification sub-module begins by parsing structured input from upstream modules and applies deterministic rule-based logic to identify direct matches based on product descriptions and predefined keyword libraries. When ambiguity exists, the sub-module leverages a classification model trained on historical assignments to suggest the most probable codes, which are then reviewed or confirmed by the compliance team. This hybrid approach ensures that cargo is tagged with the correct classification for customs, taxation, and reporting purposes, minimizing misclassification risks and regulatory friction.

The classification sub-module outputs a classification report having one or more cargo with corresponding classifications thereon, that aligns with regulatory standards, ensuring compliance and reducing the risk of penalties or revenue loss. The classification sub-module plays a critical role in supporting accurate reporting, preventing misclassification, and safeguarding tax revenue by ensuring that goods are properly classified for customs and taxation purposes.

In embodiments, the report sub-module configured to provide one or more reports using information from one or more of the sub-modules. The report sub-module synthesizes one or more data items from the one or more sub-modules, offering insights through visualizations and analytics to optimize operations, compliance, and decision-making. In embodiments, the report sub-module synthesizes operational data into actionable insights through geospatial maps, performance dashboards, and predictive analytics.

In an exemplary embodiment, the report sub-module consolidates structured data produced by all system components into a unified reporting layer, accessible through a secure web-based dashboard. The report sub-module retrieves and aggregates data via internal APIs and scheduled pipeline jobs, storing results in a reporting-optimized datastore, such as database 114. Visualization components display key performance indicators, route efficiency metrics, compliance rates, and service histories using configurable widgets, maps, and timeline views. For predictive capabilities, the module includes models trained on shipment history to forecast delays, carrier performance, or documentation issues. All reports are rendered in real time and can be exported or scheduled for delivery, enabling operational and strategic decisions to be informed by live system data.

The report sub-module is configured to processes operational data and presents it in formats such as geospatial maps, performance dashboards, and predictive analytics. The report sub-module enables decision-making by highlighting trends, performance metrics, and potential issues, supporting the optimization of operations, compliance, and strategic planning. By providing a comprehensive overview, the module enhances efficiency and helps stakeholders make informed decisions based on real-time data.

In embodiments, the fleet management sub-module configured to manage one or more carrier fleets. The fleet management sub-module receives data from one or more modules, in real-time, to monitor and optimize the performance and availability of carrier fleets, directly supporting all other components. In embodiments, the fleet management sub-module monitors vehicle performance and availability, optimizing fleet operations and enabling data-driven decisions.

In an exemplary embodiment, the fleet management submodule continuously ingests telemetry and status updates—such as GPS location, engine diagnostics, and availability flags—published by carrier vehicles and IoT sensors. A centralized service normalizes these feeds and applies predictive maintenance models (trained on historical sensor data) to flag potential equipment failures before they occur. Simultaneously, a dynamic allocation engine uses realtime availability and route assignments to rebalance vehicle resources across pending shipments, prioritizing urgent loads and minimizing idle time. All fleet events and alerts feed into the platform's event bus, ensuring other modules (e.g., routing, cargo, and reporting) can react instantly to changes in fleet status without manual intervention.

In embodiments, the fleet management sub-module tracks vehicle performance, such as fuel efficiency, maintenance status, and availability, to ensure optimal fleet operation, using one or more sensors and/or external devices. The fleet management sub-module supports data-driven decisions by providing insights into fleet utilization, helping to optimize routes, schedules, and vehicle allocation. This directly supports other modules by ensuring that transportation operations are efficient, reliable, and aligned with broader logistics objectives.

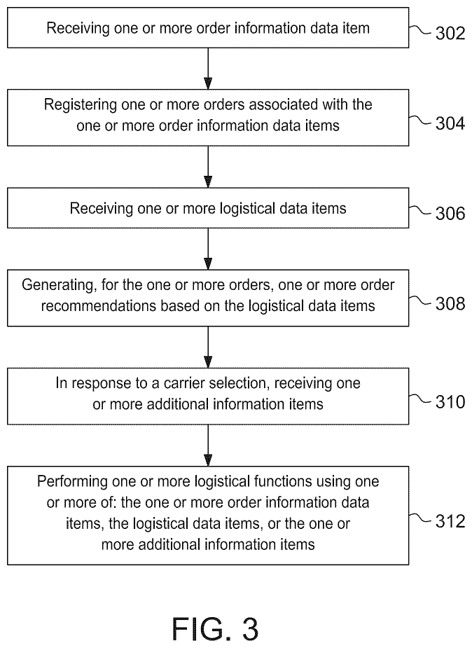

An Artificial Intelligence Virtual Assistance (AIVA) sub-module is configured to provide one or more responses to one or more users, such as user 122. In embodiments, the one or more users can interface with AIVA sub-module functionality through Interface Module 140, wherein they can ask one or more questions, and/or provide one or more digital artifacts, such as documents. In embodiments, AIVA sub-module includes one or more artificial intelligence models configured to analyze the one or more questions to determine one or more actions. In embodiments, AVA sub-modules performed method 300, as described with respect to FIG. 3.

The processing device 104, the communication device 106, the memory device 108, and the I/O interface 110 can be interconnected via a system bus. The system bus can be and/or include a control bus, a data bus, and address bus, and so forth. The processing device 104 can be and/or include a processor, a microprocessor, a computer processing unit (“CPU”), a graphics processing unit (“GPU”), a neural processing unit, a physics processing unit, a digital signal processor, an image signal processor, a synergistic processing element, a field-programmable gate array (“FPGA”), a sound chip, a multi-core processor, and so forth. As used herein, “processor,” “processing component,” “processing device,” and/or “processing unit” can be used generically to refer to any or all of the aforementioned specific devices, elements, and/or features of the processing device. While FIG. 1 illustrates a single processing device 104, the BeGo System 102 can include multiple processing devices 104, whether the same type or different types.

The memory device 108 can be and/or include computerized storage medium capable of storing electronic data temporarily, semi-permanently, or permanently. The memory device 108 can be or include a computer processing unit register, a cache memory, a magnetic disk, an optical disk, a solid-state drive, and so forth. The memory device can be and/or include random access memory (“RAM”), read-only memory (“ROM”), static RAM, dynamic RAM, masked ROM, programmable ROM, erasable and programmable ROM, electrically erasable and programmable ROM, and so forth. As used herein, “memory,” “memory component,” “memory device,” and/or “memory unit” can be used generically to refer to any or all of the aforementioned specific devices, elements, and/or features of the memory device. While FIG. 1 illustrates a single memory device 108, the BeGo System 102 can include multiple memory devices 108, whether the same type or different types.

The communication device 106 enables the BeGo System 102 to communicate with other devices and systems. The communication device 106 can include, for example, a networking chip, one or more antennas, and/or one or more communication ports. The communication device 106 can generate radio frequency (RF) signals and transmit the RF signals via one or more of the antennas. The communication device 104 can generate electronic signals and transmit the RF signals via one or more of the communication ports. The communication device 106 can receive the RF signals from one or more of the communication ports. The electronic signals can be transmitted to and/or from a communication hardline by the communication ports. The communication device 106 can generate optical signals and transmit the optical signals to one or more of the communication ports. The communication device 106 can receive the optical signals and/or can generate one or more digital signals based on the optical signals. The optical signals can be transmitted to and/or received from a communication hardline by the communication port, and/or the optical signals can be transmitted and/or received across open space by the communication device 106.

The communication device 106 can include hardware and/or software for generating and communicating signals over a direct and/or indirect network communication link. As used herein, a direct link can include a link between two devices where information is communicated from one device to the other without passing through an intermediary. For example, the direct link can include a Bluetooth™ connection, a Zigbee connection, a Wifi Direct™ connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network. An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

The BeGo System 102 can communicate with one or more network resources via the network 116. The one or more network resources can include external databases, social media platforms, search engines, file servers, web servers, or any type of computerized resource that can communicate with the BeGo System 102 via the network 116.

As described above, the BeGo System 102 can include hardware components to perform the processes described herein. In embodiments, one or more of components, hardware, and/or functionality of the BeGo System 102 can be hosted and/or instantiated on a “cloud” or “cloud service.” As used herein, a “cloud” or “cloud service” can include a collection of computer resources that can be invoked to instantiate a virtual machine, application instance, process, data storage, or other resources for a limited or defined duration. The collection of resources supporting a cloud can include a set of computer hardware and software configured to deliver computing components needed to instantiate a virtual machine, application instance, process, data storage, or other resources. For example, one group of computer hardware and software can host and serve an operating system or components thereof to deliver to and instantiate a virtual machine. Another group of computer hardware and software can accept requests to host computing cycles or processor time, to supply a defined level of processing power for a virtual machine. A further group of computer hardware and software can host and serve applications to load on an instantiation of a virtual machine, such as an email client, a browser application, a messaging application, or other applications or software. Other types of computer hardware and software are possible.

In embodiments, the components and functionality of the BeGo System 102 can be and/or include a “server” device. The term server can refer to functionality of a device and/or an application operating on a device. The server device can include a physical server, a virtual server, and/or cloud server. For example, the server device can include one or more bare-metal servers such as single-tenant servers or multiple-tenant servers. In another example, the server device can include a bare metal server partitioned into two or more virtual servers. The virtual servers can include separate operating systems and/or applications from each other. In yet another example, the server device can include a virtual server distributed on a cluster of networked physical servers. The virtual servers can include an operating system and/or one or more applications installed on the virtual server and distributed across the cluster of networked physical servers. In yet another example, the server device can include more than one virtual server distributed across a cluster of networked physical servers.

Various aspects of the systems described herein can be referred to as “information,” “content,” and/or “data.” Content and/or data can be used to refer generically to modes of storing and/or conveying information. Accordingly, data can refer to textual entries in a table of a database. Content and/or data can refer to alphanumeric characters stored in a database. Content and/or data can refer to machine-readable code. Content and/or data can refer to images. Content and/or data can refer to audio and/or video. Content and/or data can refer to, more broadly, a sequence of one or more symbols. The symbols can be binary. Content and/or data can refer to a machine state that is computer-readable. Content and/or data can refer to human-readable text.

Various of the devices in the BeGo Environment 100, including the BeGo System 102 and/or the user device 120 can provide I/O devices for outputting information in a format perceptible by a user and receiving input from the user. For example, the BeGo System 102 can communicate with the I/O devices via the I/O interface 110. The I/O devices can display graphical user interfaces (“GUIs”) generated by the BeGo System 102. The I/O devices can include a display screen such as a light-emitting diode (“LED”) display, an organic LED (“OLED”) display, an active-matrix OLED (“AMOLED”) display, a liquid crystal display (“LCD”), a thin-film transistor (“TFT”) LCD, a plasma display, a quantum dot (“QLED”) display, and so forth. The I/O devices can include an acoustic element such as a speaker, a microphone, and so forth. The I/O devices can include a button, a switch, a keyboard, a touch-sensitive surface, a touchscreen, a camera, a fingerprint scanner, and so forth. The touchscreen can include a resistive touchscreen, a capacitive touchscreen, and so forth.

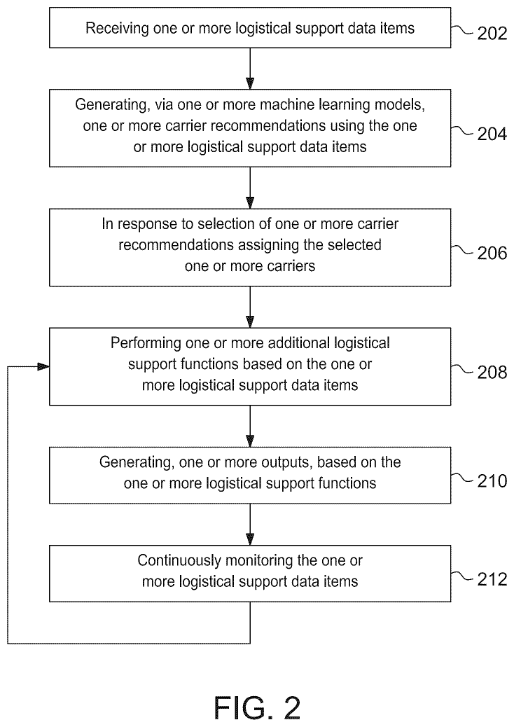

FIG. 2 shows a flowchart illustrating a method 200 for logistical support, implemented within the BeGo System 102. The method 200 is designed to optimize logistical operations by leveraging data-driven processes and machine learning models to perform various logistical functions. The method comprises multiple stages, each contributing to the seamless execution of logistical tasks. In embodiments, one or more modules, such as BeGo Module 142, and/or one or more sub-modules thereof, operate to execute one or more aspects of method 200. In embodiments, method 200 is iterative, and receives data in real-time, in batches, or otherwise as needed for performance thereof. In embodiments, method 200 is iterative, and/or recursive, and as such, receives the one or more logistical support data items, in real-time, at differing times, and/or as needed.

The first stage, receiving one or more logistical support data items 202, involves collecting and analyzing data related to shipments and carriers. The logistical support data items may include cargo details such as weight, dimensions, and handling requirements, as well as carrier-specific information, including vehicle capacity, availability, and historical performance metrics. This stage ensures that all relevant data is gathered and prepared for subsequent processing. The data may be sourced from relational and non-relational Databases 114, geolocation systems, sensors, and user inputs, ensuring a comprehensive dataset for analysis.

The second stage, generating, via one or more machine learning models, one or more carrier recommendations using the one or more logistical support data items 204, utilizes advanced algorithms to generate optimal pairing recommendations between shippers and carriers. The machine learning models analyze the resolved data to minimize constraints such as transportation costs, delays, and empty cargo mileage while maximizing efficiency and compliance. These recommendations are tailored to meet operational and regulatory requirements. For example, optimization algorithms such as mixed-integer linear programming may be employed to identify the most efficient pairings, while rule-based validation engines ensure compliance with shipment requirements established by regulations.

The third stage, in response to selection of one or more carrier recommendations, performing, in real-time, one or more logistical support functions 206, involves the execution of logistical tasks based on the selected recommendation. Once a user or system selects a recommendation, the corresponding logistical functions are performed. These functions may include, one or more of: matching one or more cargo shippers with one or more cargo carriers, automatically generate one or more documents needed for cargo shipping, automating financial transactions between the one or more cargo shippers and the one or more cargo carriers, providing funding solutions for the one or more cargo carriers, providing one or more medical services and/or insurance services to the one or more cargo carriers, analyzing and preventing one or more compliance issues, analyzing and valuing goods in cargo, generating standardized cargo information for compliance when shipping across one or more jurisdictional borders, evaluating one or more cargo details, generating and evaluating one or more routes for the one or more cargo carriers, classifying one or more cargo, providing one or more reports using information from one or more of the sub-modules, and/or managing one or more carrier fleets. The real-time nature of this stage ensures that logistical operations remain adaptive to dynamic conditions and user inputs.

The final stage, generating one or more outputs, based on the one or more logistical support functions 208, ensures that the results of the logistical functions are communicated to relevant stakeholders. This stage may involve generating reports, updating system records, or notifying users of the completed tasks. The outputted information items provide actionable insights and ensure transparency in the logistical process. For instance, the system may produce detailed financial documents, compliance reports, or route optimization metrics, which can be accessed through user interfaces or APIs. In embodiments, the one or more outputs vary based on the one or more logistical support functions selected.

For example, a logistical support function can be automatic generation of one or more documents needed for cargo shipping, as described in the regulatory sub-module of BeGo Module 142. In embodiments, the one or more documents can be a Carta Porte, or Bill of Lading, generated using the one or more logistical support data items. In embodiments, a check is performed to determine if all required parameters are present, and if not one or more missing required parameters are requested. Once all required parameters are present, the one or more documents are generated, and stored in a user account within BeGo system 102, such as by Storage Module 144 in databases 114. In embodiments, the one or more documents can be provided to one or more internal, or external, users via notifications, alerts, email, SMS, mms, etc., and/or can be accessed via BeGo system 102 such as through a mobile interface, web interface, etc.

A logistical support function can be automating one or more financial transactions between the one or more cargo shippers and the one or more cargo carriers, as described by the invoice sub-module. In embodiments, the one or more logistical support data items is one or more of shipper information, receiver information, and/or one or more transaction details, which are utilized to generate one or more digital invoices. In embodiments, the one or mor digital invoices comply with one or more jurisdictional regulatory requirements, such as Comprobantes Fiscal Digital por Internet (CFDI). Once generated, the one or more digital invoices are provided to one or more internal, or external, users via notifications, alerts, email, SMS, mms, etc., and/or can be accessed via BeGo system 102 such as through a mobile interface, web interface, etc. Additionally, the one or more digital invoices can be integrated with one or more external systems such as a payment gateway, allowing users of BeGo System 102 to pay invoices upon receipt.

A logistical support function can be providing one or more medical services and/or insurance services to the one or more cargo carriers, as described in the medical sub-module. In embodiments, the one or more logistical support data items includes a unique user identifier, such as a Social Security Number, Tax Identifier Number, CURP, specific to a jurisdiction. The unique identifier is checked against one or more regulatory databases, such as an Anti-money Laundering Database (AML), wherein if the unique identifier is present, no medical or insurance services are provided.

If the unique identifier is not present in the one or more regulatory databases, personal information such as name, birth date, gender, address, etc., are extracted from the one or more logistical data items, and one or more insurance quotes, including one or more prices and one or more terms, are provided. In embodiments, one or more insurance quotes are accepted by the user. Upon acceptance, a user accesses a payment system to pay the quoted one or more prices, and an insurance policy is generated. Once generated, the insurance policy is provided to internal, or external, users via notifications, alerts, email, SMS, mms, etc., and/or can be accessed via BeGo system 102 such as through a mobile interface, web interface, etc.

In addition to the above one or more additional logistical support functions can be as follows: A logistical support function can be analyzing and preventing one or more compliance issues, as described in the compliance sub-module; A logistical support function can be cargo valuation, as described in the valuation sub-module; A logistical support function can be generating standardized cargo information for compliance when shipping across one or more jurisdictional borders, as described in the customs sub-module; A logistical support function can be evaluating cargo details, as described in the cargo evaluation sub-module; A logistical support function can be generating, updating, and/or evaluating routes, as described in the routing sub-module; A logistical support function can be classifying cargo, as described in the classification sub-module; A logistical support function can be providing one or more reports for using in one or more sub-modules, or for display to one or more users, as described in the report sub-module; A logistical support function can be managing one or more carriers, or fleets of carriers, as described in the fleet management sub-module; and/or A logistical support function can be providing funding solutions for the one or more cargo carriers, as described in the funding sub-module.

The iterative nature of method 200 allows the approach to adapt to real-time data inputs and changing operational conditions. By continuously refining the processes based on new data, the method ensures that logistical operations remain efficient, compliant, and responsive to dynamic environments.

FIG. 3 shows a flowchart illustrating a method 300 for placing one or more orders within BeGo System 102. In embodiments, method 300 comprises multiple stages, each contributing to the seamless execution of logistical tasks. In embodiments, method 300 is performed by BeGo Module 142, and/or one or more of the sub-modules thereof.

Method 300 begins at step 302, with one or more users, such as user 122 accessing BeGo system 102, using one or more interfaces, and providing one or more order information data items. In embodiments, step 302 receives one or more order information data items, such as fiscal data, cargo details, order details, and/or one or more documents. In embodiments, BeGo System 102, through BeGo Module 142, or a sub-module thereof, performs one or more validation, and/or compliance checks on the information to determined completeness and/or correctness, and takes the appropriate action, such as requesting completion, or correction, if the information is not correct.

At step 304, once the one or more order information items are entered and validated they are entered registered for order creation. In embodiments, registration includes storing the information items into a data storage, such as database 114, for use in BeGo system, and/or by BeGo module 142.

At step 306, one or more logistical data items are received in order to create one or more orders. In embodiments, the one or more logistical data items are one or more of: an order pickup address, an order delivery address, a date for pickup, a time for pickup, one or more of the logistical support data items from step 202 of method 200 and/or a selection of one or more required documents, such as a bill of lading, or Carta Porte.

At step 308, the one or more logistical data items are utilized to generate one or more carrier recommendations. In embodiments, the one or more carrier recommendations includes one or more recommendations of: one or more drivers, one or more shippers, and/or one or more containers. In embodiments, recommendations are generated the same as, or similar to, the one or more carrier recommendations of step 204 of method 200. In embodiments, the user can select one of the one or more carrier recommendations for the one or more orders.

At step 310, in response to selection of one of the one or more carrier recommendations, one or more additional information items are received with respect to the one or more orders. In embodiments, the user can enter additional information associated with the one or more orders. In embodiments, the additional information includes one or more pickup warehouse personnel responsible for the one or more orders, such as a name, phone number, email address, a unique identifier, Company name, reference, etc. In embodiments, the additional information includes one or more destination warehouse personnel responsible for the one or more orders, such as a name, phone number, email address, a unique identifier, Company name, reference, etc. In embodiments, the one or more additional information includes one or more types of goods for the one or more orders, one or more weights, if a container is being used the type of container (i.e. 20, 40, 40 HQ, etc.), a load description (i.e. quantity of goods, types of units, etc.), a designation of hazardous/non-hazardous materials, a total price, a currency requirement, a recipients billing information, etc.

At step 312, all information received is saved and utilized in one or more logistical support functions. Specifically, step 312 includes performing one or more logistical functions using one or more of: the one or more order information data items, the logistical data items, or the one or more additional information items. In embodiments, the one or more logistical support functions include one or more of the functions performed in method 200, and/or by BeGo Module 142, and/or any sub-module thereof. In embodiments, FIG. 3 illustrates a more detailed view of steps 202-206 of method 200.

Method 400 begins at step 402 with a user providing one or more questions, and/or digital artifacts to the AIVA. In embodiments, the one or more questions are input, using I/O interface, and can be text, image, Natural Language, and/or any other format perceptible by a computing device. In embodiments, the one or more digital artifacts are one or more digital files, such as images, documents, sound files, etc.

At step 404, the AIVA analyzes the one or more questions and/or digital artifacts to determine if a question is present which needs an answer, or one or more actions need to be taken, which results in a decision at step 406.

At step 408, the AIVA has determined an action needs to be taken, and the AIVA determines one or more modules needed to perform the one or more actions. In embodiments, the one or more modules can be BeGo module 142 and/or any sub-module of BeGo module 142. In embodiments, once one or more modules is determined AIVA can assist the user in utilizing the module to complete the action at step 410. For example, if it is determined that the customs sub-module is needed to perform an action AIVA can assist user in utilizing the customs module by gathering and automatically imputing data, and/or providing explanations to the user as to how to utilize the sub-module.

At step 412, the AIVA has determined that one or more questions needs to be answered, and determines one or more answers to the one or more questions. In embodiments, the AIVA utilizes both internal and external data sources to provide the one or more answers. Once the one or more answers are determined by the AIVA they are output at step 414. In embodiments, the one or more answers can be output via a GUI, and/or one or more notifications, such as email, text, graphical alert, etc.

As used in the description herein and throughout the claims that follow, “a”, “an”, and “the” include plural references unless the context clearly dictates otherwise. Also, as used in the description herein and throughout the claims that follow, the meaning of “in” includes “in” and “on” unless the context clearly dictates otherwise. While the above is a complete description of specific examples of the disclosure, additional examples are also possible. Thus, the above description should not be taken as limiting the scope of the disclosure which is defined by the appended claims along with their full scope of equivalents.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements. As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

It should be understood, of course, that the foregoing relates to exemplary embodiments of the disclosure and that modifications can be made without departing from the spirit and scope of the disclosure as set forth in the following claims.

It should be understood, of course, that the foregoing relates to exemplary embodiments of the invention and that modifications may be made without departing from the spirit and scope of the invention as set forth in the following claims.

What is claimed is:

1. A system for logistical support, comprising:
a processing device;
a communication device coupled to the processing device and configured to receive logistical support data items from one or more data sources;
a memory device coupled to the processing device and having stored thereon:
an interface module configured to provide at least one of a graphical user interface and an application programming interface for receiving from a user device the logistical support data items and for outputting to the user device one or more outputs;
a storage module configured to store the logistical support data items; and
a Logistical Support module comprising a plurality of sub modules, each sub module comprising logic executable by the processing device, the plurality of sub modules comprising:
a cargo sub module configured to match one or more shippers of cargo with one or more carriers of cargo based on the logistical support data items;
a regulatory sub module configured to generate one or more regulatory documents based on the logistical support data items to support compliance with governmental requirements;
an invoice sub module configured to generate and transmit one or more invoices for financial transactions between the one or more shippers and the one or more carriers based on the logistical support data items;
a funding sub module configured to determine and provide funding solutions to the one or more carriers based on the logistical support data items and one or more invoices generated by the invoice sub module;
a medical sub module configured to enroll one or more carriers in one or more insurance plans and to provide medical services based on the logistical support data items;
a compliance sub module configured to analyze the logistical support data items and to detect and prevent compliance issues by identifying anomalies in regulatory documents and invoices;
a valuation sub module configured to determine monetary valuations of goods in cargo based on the logistical support data items and external benchmark data;
a customs sub module configured to transform and format the logistical support data items into standardized customs information for cross border shipment compliance;
a cargo evaluation sub module configured to classify cargo details and to identify hazardous materials and specific handling requirements;
a routing sub module configured to generate and evaluate routes for the one or more carriers based on geospatial data and risk assessments;
a classification sub module configured to classify cargo using standardized classification codes based on the logistical support data items;
a report sub module configured to synthesize operational and compliance data into visual reports; and
a fleet management sub module configured to monitor and optimize one or more carrier fleets based on real time telemetry and status data;
wherein the processing device is configured to:
receive the logistical support data items via the interface module;
store the logistical support data items via the storage module;
generate, via one or more machine learning models executed by the processing device, one or more pairing recommendations between the one or more shippers and the one or more carriers based on the logistical support data items;
in response to selection of one of the pairing recommendations, invoke at least one of the plurality of sub modules to perform the corresponding logistical support function in real time; and
output via the interface module the one or more outputs including results of the performed logistical support function.

2. The system of claim 1, wherein the cargo sub module generates pairing recommendations using:
a mixed integer linear programming model; or
predictive scores from a supervised machine learning model.

3. The system of claim 1, wherein the regulatory sub module is configured to:
automatically generate a Carta Porte or a Bill of Lading;
validate each generated regulatory document against a validation schema; and
fetch the validation schema from an external regulatory repository.

4. The system of claim 1, wherein the funding sub module employs:
a dynamic scoring model combined with a rules-based trust engine to compute a suggested funding amount; and
an interface with one or more external financial platforms to disburse funds to the one or more carriers.

5. The system of claim 1, wherein the medical sub-module integrates with:
telemedicine services to schedule virtual consultations with remote medical professionals; and
wearable devices to monitor carrier physiological indicators in real time.

6. The system of claim 1, wherein the compliance sub module employs:
an unsupervised learning algorithm trained on historical transaction data to detect anomalies in invoices and regulatory documents; and
reinforcement learning with human feedback to refine detection accuracy over time.

7. The system of claim 1, wherein the valuation sub module applies statistical anomaly detection to:
compare declared cargo values against internal benchmarks synchronized with external customs valuation databases; and
flag undervaluation cases for manual review.

8. The system of claim 1, wherein the routing sub module is further configure to:
compute a dynamic crime risk index for each candidate route segment by analyzing, one or more of: one or more historical crime statistics, one or more time-of-day sensitivity conditions, one or more environmental conditions.

9. A method for providing logistical support, performed by a computing system comprising at least one processor, a communication interface, and a non-transitory storage medium, the method comprising:
receiving, via the communication interface, one or more logistical support data from one or more data sources;
generating, via one or more machine learning models executed by the processor, one or more carrier pairing recommendations based on the one or more logistical support data;
in response to selection of at least one of the carrier pairing recommendations by a user device, performing, in real time, at least one logistical support function selected from the group consisting of:
generating, using the one or more logistical support data, one or more regulatory shipment documents;
generating, using the one or more logistical support data, one or more invoices for shipping transactions;
determining and providing, using the one or more logistical support data, one or more funding solutions for the at least one carrier pairing recommendation;
enrolling, using the one or more logistical support data, the at least one carrier pairing recommendation in insurance plans and providing medical services;
detecting and preventing one or more compliance issues by analyzing, using the one or more logistical support data;
determining, using the one or more logistical support data, one or more monetary valuations of one of more goods;
generating, using the one or more logistical support data, one or more standardized customs information for cross-border shipment compliance;
evaluating, using the one or more logistical support data, the one or more goods to identify hazardous materials and handling requirements;
generating and evaluating, using the one or more logistical support data, one or more shipment routes based on geospatial data and risk assessments;
classifying, using the one or more logistical support data, the one or more goods according to one or more standardized classification codes;
providing visual reports comprising performance metrics and predictive analytics; and
monitoring and optimizing carrier fleet performance based on the one or more logistical support data; and
outputting, via the communication interface, one or more outputs comprising results of the performed logistical support function to the user device.

10. The method of claim 9, wherein the logistical support data comprising at least one of cargo details, vehicle capacity, geolocation data, sensor data, and user inputs.

11. The method of claim 9, wherein the one or more machine learning models is one or more of: a mixed integer linear programming model; or a supervised machine learning model.

12. The method of claim 9, wherein generating regulatory shipment documents comprises:
automatically generating at least one of a Carta Porte, or a Bill of Lading; and
validating each generated document against a stored validation schema.

13. The method of claim 9, wherein determining and providing funding solutions comprises:
computing a suggested funding amount using a dynamic scoring model combined with a rules-based trust engine; and
disbursing the suggested amount via a financial platform.

14. The method of claim 9, wherein enrolling carriers in insurance plans and providing medical services comprises:
verifying a distinct user identifier against an anti-money laundering database; and
generating insurance policy documents upon successful verification.

15. The method of claim 9, wherein detecting and preventing one or more compliance issues comprises:
detecting one or more anomalies in regulatory or financial data, using an unsupervised learning algorithm trained on historical transaction data, wherein a detection accuracy is refined using reinforcement learning with feedback.

16. The method of claim 9, wherein generating one or more standardized customs information comprises:
mapping the logistical support data to jurisdiction-specific electronic formats; and
mapping the logistical support data to one or more codes based on a shipment destination.

17. The method of claim 9, wherein generating and evaluating one or more shipment routes comprises:
computing a dynamic crime risk index for each route segment based on historical crime statistics;
computing the dynamic crime risk index based on time-of-day sensitivity conditions; and
computing the dynamic crime risk index based on environmental conditions.
